(19) United States
(12) Patent Application Publication
(10) Pub. No.: US 2006/0241299 A1
(43) Pub. Date: Oct. 26, 2006

(54) Process for making spirolactone compounds

(76) Inventors: Ralph P Volante, Cranbury, NJ (US); Steven A. Weissman, Short Hills, NJ (US); Takehiko Iida, Okazaki (JP); Yuhei Yamamoto, Chicago, IL (US); Hiroki Sato, Okazaki (JP); Kenji Maeda, Okazaki (JP); Naotaka Sawada, Okazaki (JP); Toshiaki Mase, Okazaki (JP)

Correspondence Address: MERCK AND CO., INC, P O BOX 2000, RAHWAY, NJ, 07065-0907, US

(21) Appl. No.: 10/550,136

(22) PCT Filed: May 14, 2004
(86) PCT No.: PCT/US04/15051

§ 371 Date: Sep. 21, 2005

Related U.S. Application Data
(60) Provisional application No. 60/471,680, filed on May 19, 2003.

(51) Int. Cl. C07D 491/10

(52) U.S. Cl. 546/16

(57) ABSTRACT

This invention relates to a process for making spirolactone compounds of general formula I, having an improved IA/IB ratio, according to the following shceme.

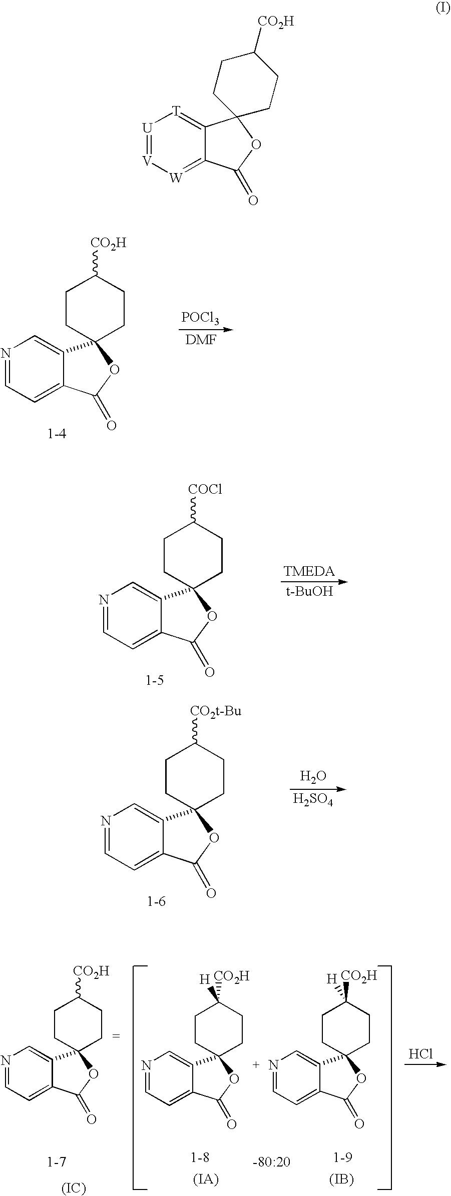

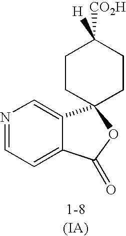

### BACKGROUND OF THE INVENTION

The present invention relates to a process for the preparation of the spirolactones of formula I. 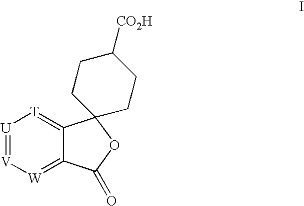
The compounds of formula I are intermediates useful for the preparation of the spirolactone compounds of formula II.

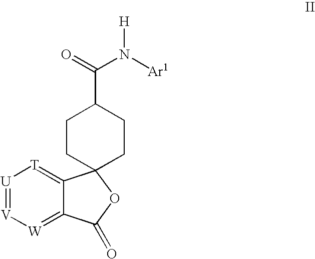

The compounds of formula II, along with their use as NPY5 antagonists for treating bulimia, obesity or diabetes, were disclosed in U.S. Pat. No. 6,335,345, which is incorporated by reference herein in its entirety, and in WO 01/14376 (published on Mar. 2, 2001). The compounds of formula II are also useful as agents for the treatment of various diseases related to NPY, including, but not limited to, cardiovascular disorders, such as hypertension, nephropathy, heart disease, vasospasm, arteriosclerosis and the like, central nervous system disorders, such as bulimia, depression, anxiety, seizure, epilepsy, dementia, pain, alcoholism, drug withdrawal and the like, metabolic diseases such as obesity, diabetes, hormone abnormality, hypercholesterolemia, hyperlipidemia and the like, sexual and reproductive dysfunction, gastrointestinal disorder, respiratory disorder, inflammation or glaucoma, and the like.

U.S. Pat. No. 6,335,345 and WO 01/14376, describe a process for preparing the compounds of formula II from the spirolactone of formula I.

U.S. Pat. No. 6,388,077 and U.S. Ser. No. 60/352,451 describe processes for preparing the compounds of formula I. However, a large number of synthetic transformations are required (the longest linear sequence being about 7 steps) with an overall yield between about 15-20%.

Separation of the cis and trans spirolactone acids IA and IB in the previous syntheses resulted in the loss of all of the material prepared as the wrong enantiomer. The present invention relates to a process for enriching the trans:cis ratio of the spirolactone acid of formula I comprising the spirolactone acid mixture, IC, shown on page 3. The process leads to an increase in the amount of trans spirolactone acid IA in the spirolactone acid mixture IC relative to the amount of cis spirolactone acid IB in the spirolactone acid mixture IC. This enrichment process leads to a higher yield of the trans spirolactone acid IA.

Processes for the preparation of organolithium reagents, 3-benzylpicolinic and 3-benzylisonicotinic acids, as well as lactone ring formation, are described in Synthetic Communications, 20 (17), pp. 2623-2629 (1990). Processes for the ortho-lithiation of N-propenylbenzamides and N-propenyl-o-toluamides are described in J. Org. Chem., vol. 57, pp. 2700-2705 (1992). Reactions of alcohols and ketenes to give esters are disclosed in Tidwell, T. T: “Ketenes” John Wiley & Sons: New York, N.Y., 1995, p. 592-597. The use of hindered alcohols to de-racemize prochiral carboxylic acids is described in Larsen, R. D. et al., J. Am. Chem. Soc. 1989, 111, 7650; Calmes, M. et al., Tetrahedron: Asymmetry 2002, 13, 293; and Calmes, M. et al., Tetrahedron, 1997, 40, 13719.

### SUMMARY OF THE INVENTION

The present invention provides a process for preparing compounds of structural formula I.

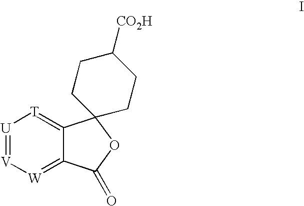

The process involves anion formation, such as ortho-lithiation, of an aromatic compound followed by reaction with an ester-substituted cyclohexanone, hydrolysis and lactone ring formation. The resulting spirolactone acid is converted to an acid halide, which is subsequently converted to a sterically hindered ester via a ketene intermediate. The sterically hindered ester is hydrolyzed to give the desired spirolactone of formula IC, predominately in the trans form (IA). Crystallization of spirolactone IC, or a salt thereof, and separation gives isomers IA and IB, or a salt thereof, in highly pure form.

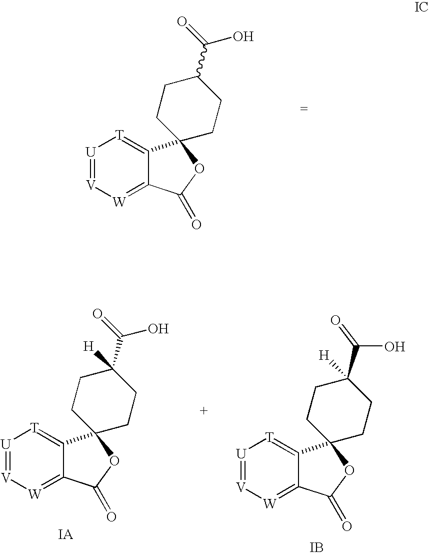

Individually reacting the separated spirolactones of formula IA or IB with an amine of the formula H₂NAr¹ gives the corresponding spirolactone amide IIA or IIB, as shown in general Scheme 1.

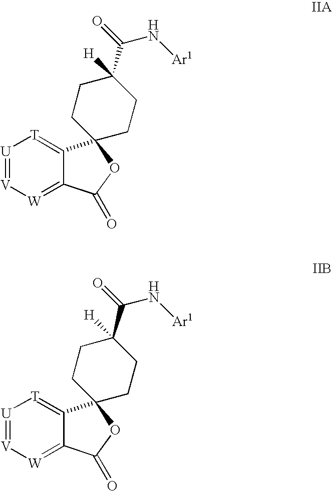

In Scheme 1, the reaction of the 4-ester substituted cyclohexanone B with the ortho-lithiated aromatic compound A is followed by ester hydrolysis and lactone ring formation to give the spirolactone acid IC, as a mixture with a ratio of approximately 1:1 IA to IB. The spirolactone acid IC is then activated by conversion to acid halide E, which is subsequently converted to a sterically hindered ester F, via a ketene intermediate, by treatment with a sterically hindered alcohol R³OH. The resulting sterically hindered ester F is then hydrolyzed to give spirolactone acid IC, as a mixture of spirolactone acids of formula IA and IB with a ratio of approximately 80:20 trans (IA) to cis (IB). The mixture of IA and IB may be separated via crystallization by treatment of the mixture with an acid, to form a salt of IB, and subsequently separating IA and IB. The trans spirolactone acids IA and IB may then be individually reacted with H₂NAr¹ to give compounds of formula IIA and IIB.

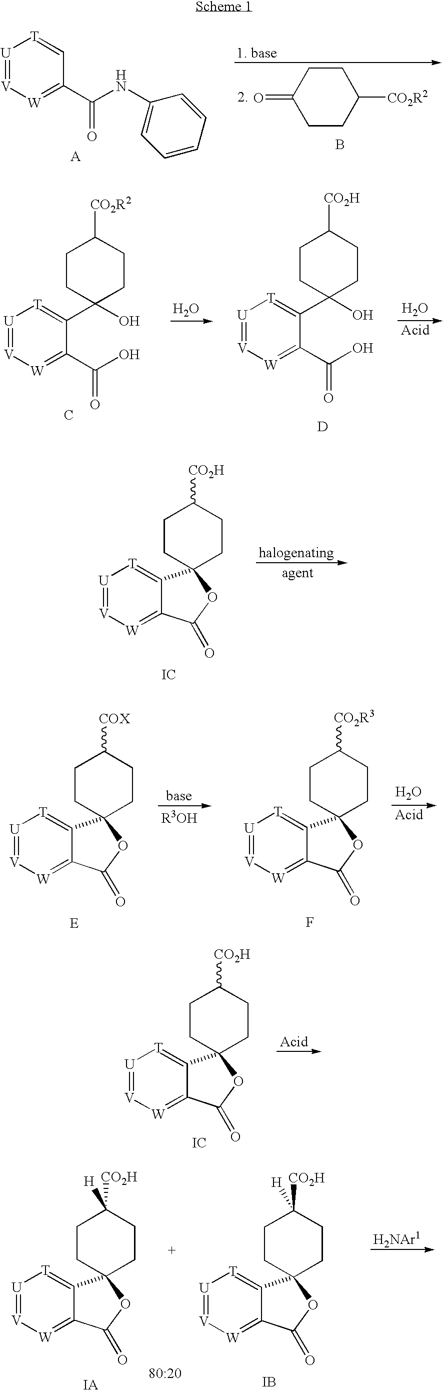

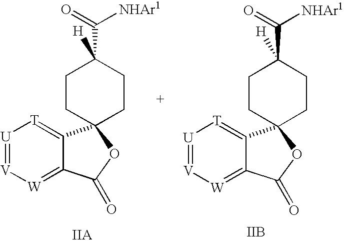

### DETAILED DESCRIPTION OF THE INVENTION

By this invention, there is provided a process for the preparation of a compound of structural formula IC, or a salt thereof, 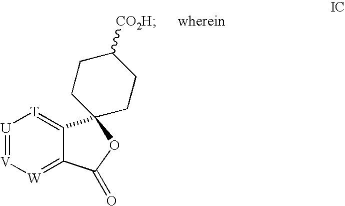
T, U, V and W are each independently selected from the group consisting of:
(1) nitrogen, and
(2) methine,
wherein the methine group is unsubstituted or optionally substituted with a substituent selected from the group consisting of:
   (a) halogen,
   (b) lower alkyl,
   (c) hydroxy, and
   (d) lower alkoxy, and
      wherein at least two of T, U, V, and W are methine;
      comprising the steps of:
(a) forming an spirolactone acid halide of formula E 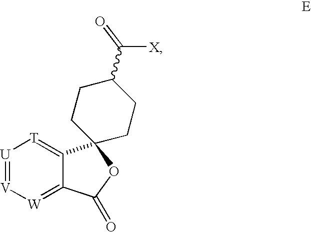
wherein X is chlorine or bromine, and T, U, V, and W are as defined above, by treating the compound of formula IC with a halogenating agent in a solvent;
(b) forming a spirolactone ester of formula P 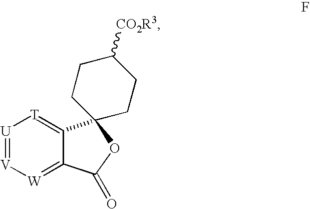
wherein R³ is selected from the group consisting of tert-butyl, methyl cyclohexyl, methyl cyclopentyl, and neopentyl, and T, U, V and W are as defined above, by treating the spirolactone acid halide of formula E with a base and an alcohol in a solvent;
(c) forming a spirolactone acid of formula IC 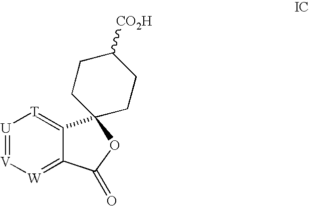
wherein T, U, V and W are defined as above, by hydrolyzing the spirolactone ester of formula F with an aqueous acid; and
(d) isolating the resulting product.

In one embodiment of the present invention, the process comprises increasing the amount of trans isomer IA 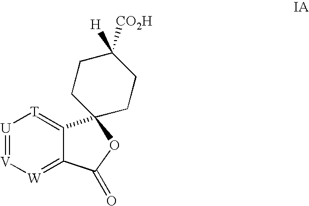
in the compound of structural formula IC 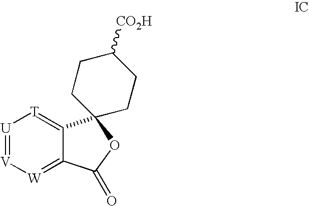
relative to the amount of cis isomer IB 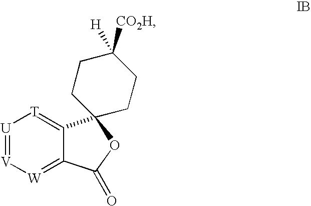
in the compound of structural formula IC,
wherein T, U, V and W are each independently selected from the group consisting of:
(1) nitrogen, and
(2) methine,
wherein the methine group is unsubstituted or optionally substituted with a substituent selected from the group consisting of:
   (a) halogen,
   (b) lower alkyl,
   (c) hydroxy, and
   (d) lower alkoxy, and
wherein at least two of T, U, V, and W are methine.

In another embodiment of the present invention, T, V and W are methine, wherein the methine group is unsubstituted or optionally substituted with a substituent selected from the group consisting of
(a) halogen,
(b) lower alkyl,
(c) hydroxy, and
(d) lower alkoxy; and
   U is nitrogen.

In a class of this embodiment, T, V and W are unsubstituted methine; and U is nitrogen.

In another embodiment of the present invention, T, U, V and W are methine, wherein the methine group is unsubstituted or optionally substituted with a substituent selected from the group consisting of
(a) halogen,
(b) lower alkyl,
(c) hydroxy, and
(d) lower alkoxy.

In one class of this embodiment, the methine group is unsubstituted or optionally substituted with halogen.

In another embodiment of the present invention, the solvent in step (a) is selected from the group consisting of chloroform, ethyl acetate, tetrahydrofuran, dimethoxyethane, diglyme, 2-methyl tetrahydrofuran, 1,4-dioxane and diethoxymethane. In a class of this embodiment, the solvent in step (a) is tetrahydrofuran.

In another embodiment of the present invention, the halogenating agent in step (a) is selected from the group consisting of phosphorus oxychloride, oxalyl chloride, phosphorus trichloride, phosphorus tribromide, thionyl chloride, thionyl bromide and oxalyl bromide. In a class of this embodiment, the halogenating agent in step (a) is phosphorus oxychloride. In a subclass of this class, the amount of phosphorus oxychloride is between about 0.7 equivalents to about 2.0 equivalents relative to spirolactone acid IC. In another subclass of this class, the amount of phosphorus oxychloride is about 1.15 equivalents relative to spirolactone acid IC. In another subclass of this class, the amount of phosphorus oxychloride is about 1.05 equivalents relative to spirolactone acid IC.

In another embodiment of the present invention, the spirolactone acid halide of formula E in step (a) is a spirolactone acid chloride.

In another embodiment of the present invention, the reaction of step (a) further comprises a catalyst. In a class of this embodiment, the catalyst is dimethyl formamide. In a subclass of this class, the amount of dimethyl formamide is between about 0.2 equivalents to about 5 equivalents relative to spirolactone acid of formula IC. In another subclass of this class, the amount of dimethyl formamide is about 1 equivalent relative to spirolactone acid of formula IC.

In another embodiment of the present invention, the reaction of step (a) is run at a temperature between about 20° C. to about 80° C. In a class of this embodiment, the reaction of step (a) is run at a temperature of about 40° C. In a subclass of this class, the reaction of step (a) is run at a temperature of about 40° C. for about 2 hours.

In another embodiment of the present invention, the base of step (b) is selected from the group consisting of N,N,N′,N′-tetramethylethylenediamine, triethyl amine, N,N-diisopropylethyl amine, N,N-dimethylethyl amine, pyridine, collidine, 1,8-diazabicyclo[5.4.0]undec-7-ene, N-methylmorpholine, and N,N,N′,N′-tetramethyl-1,6-hexanediamine. In a class of this embodiment, the base of step (b) is N,N,N′,N′-tetramethylethylene-diamine. In a subclass of this class, the amount of N,N,N′,N′-tetramethylethylene-diamine is between about 1 equivalent to about 10 equivalents relative to spirolactone ester of formula F. In another subclass of this class, the amount of N,N,N′,N′-tetramethyl-ethylenediamine is about 3.5 equivalents relative to spirolactone ester of formula F.

In another embodiment of the present invention, the alcohol of step (b) is selected from the group consisting of tert-butyl alcohol, methyl cyclohexanol, methyl cyclopentanol, and neopentyl alcohol. In a class of this embodiment, the alcohol of step (b) is tert-butyl alcohol. In a subclass of this class, the amount of tert-butyl alcohol is between about 1 equivalent to about 10 equivalents relative to spirolactone ester of formula F. In another subclass of this class, the amount of tert-butyl alcohol is about 1.5 equivalents relative to spirolactone ester of formula F.

In one embodiment of the present invention, the solvent in step (b) is selected from the group consisting of tetrahydrofuran, dimethoxyethane, diglyme, 2-methyl tetrahydrofuran, 1,4-dioxane and diethoxymethane. In a class of this embodiment, the solvent in step (b) is tetrahydrofuran.

In another embodiment, the reaction of step (b) further comprises a salt. In a class of this embodiment, the salt is selected from the group consisting of lithium bromide, lithium chloride, lithium iodide, lithium perchlorate and lithium tetrafluoroborate. In a subclass of this class, the salt is lithium chloride. In a subclass of this subclass, the amount of lithium chloride is between about 0.5 equivalents to about 5 equivalents relative to spirolactone ester of formula F. In another subclass of this subclass, the amount of lithium chloride is about 1 equivalent relative to spirolactone ester of formula F.

In another embodiment of the present invention, the reaction of step (b) is run at a temperature between about 20° C. to about 80° C. In a class of this embodiment, the reaction of step (b) is run at a temperature of about 40° C. In a subclass of this class, the reaction of step (b) is run at a temperature of about 40° C. for about 2 hours to about 24 hours. In another subclass of this class, the reaction of step (b) is run at a temperature of about 40° C. for about 19 hours.

In another embodiment of the present invention, the aqueous acid of step (c) is selected from the group consisting of sulfuric acid, hydrochloric acid, hydrobromic acid, phosphoric acid and formic acid. In a class of this embodiment, the aqueous acid of step (c) is sulfuric acid.

In another embodiment of the present invention, the hydrolysis of step (c) is run at a temperature between about 20° C. and about 100° C. In a class of this embodiment, the hydrolysis of step (c) is run at a temperature of about 50° C. In a subclass of this class, the hydrolysis of step (c) is run at a temperature of about 50° C. for about 2 hours.

In another embodiment of the present invention, the product of step (d) is isolated by adjusting the pH of the solution of step (c) to between about 0 and 4 with a base and extracting the reaction mixture to afford the compound IC. In a subclass of this class, the base is sodium hydroxide. In another subclass, the pH of the solution of step (c) is adjusted to between about 2 to about 3. In a subclass of this subclass, the pH of the solution of step (c) is adjusted to about 2.4.

By this invention, there is further provided a process for the preparation and separation of a spirolactone of formula IA, or a salt thereof, and a spirolactone of formula IB, or a salt thereof, 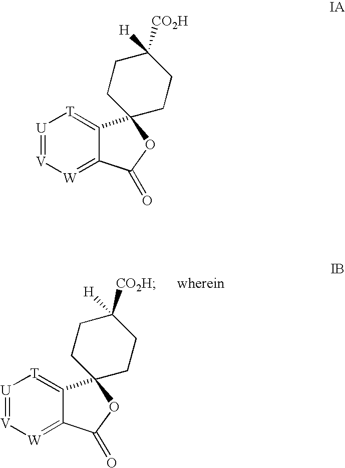
T, U, V and W are each independently selected from the group consisting of
(1) nitrogen, and
(2) methine,
wherein the methine group is unsubstituted or optionally substituted with a substituent selected from the group consisting of
   (a) halogen,
   (b) lower alkyl,
   (c) hydroxy, and
   (d) lower alkoxy, and
      wherein at least two of T, U, V, and W are methine;
      comprising the steps of
(e) adding a solvent to the compound of formula IC, 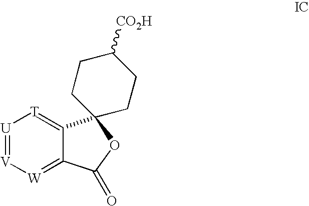
wherein T, U, V and W are as defined above, to form a mixture;
(f) adding an acid to the mixture of step (e) to form a mixture; and
(g) aging the mixture of step (f) for a time and under conditions effective to afford the compound IA 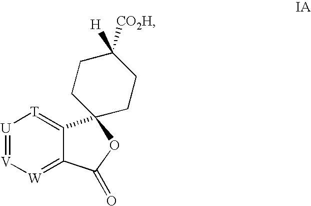
wherein T, U, V and W are as defined above, or a salt thereof.

In one embodiment of the present invention, T, V and W are methine, wherein the methine group is unsubstituted or optionally substituted with a substituent selected from the group consisting of
(a) halogen,
(b) lower alkyl,
(c) hydroxy, and
(d) lower alkoxy; and
   U is nitrogen.

In a class of this embodiment, T, V and W are unsubstituted methine; and U is nitrogen.

In another embodiment of the present invention, T, U, V and W are methine, wherein the methine group is unsubstituted or optionally substituted with a substituent selected from the group consisting of
(a) halogen,
(b) lower alkyl,
(c) hydroxy, and
(d) lower alkoxy.

In one class of this embodiment, the methine group is unsubstituted or optionally substituted with halogen.

In another embodiment of this invention, the solvent of step (e) is selected from the group consisting of dimethoxyethane, acetonitrile, tetrahydrofuran, or a mixture thereof. In a class of this embodiment, the solvent of step (e) is tetrahydrofuran. In another class of this embodiment, the solvent of step (e) is acetonitrile.

In another embodiment of this invention, the acid of step (f) is selected from the group consisting of hydrochloric acid, hydrobromic acid, tartaric acid, methane sulfonic acid, toluene sulfonic acid, succinic acid, and sulfuric acid. In a class of this embodiment, the acid of step (f) is hydrochloric acid. In another embodiment of this invention, the step (g) is aged at a temperature of about 10° C. to 60° C. In a class of this embodiment, step (g) is aged for a period between about 1 hour to about 48 hours. In a subclass of this class, step (g) is aged at a temperature of about 25° C. for about 3 hours. In another embodiment of this invention, the process further comprises step (h) of isolating the compound of formula IA, or a salt thereof. In a class of this embodiment, the compound of formula IA is isolated by filtering and concentrating the filtrate to give a slurry. In a subclass of this class, the slurry is diluted with a solvent and aged for a time and under conditions to give the compound of formula IA. In another subclass of this class, the slurry is diluted with hexane and aged for about 20 hours at about 0° C. In a subclass of this subclass, the compound of formula IA is isolated by filtering the slurry to give the product. In another subclass of this class, the slurry is concentrated, diluted with acetonitrile and aged for a time and under conditions to give the compound of formula IA.

By this invention, there is also provided a process for the preparation of a compound of structural formula IC, or a salt thereof, 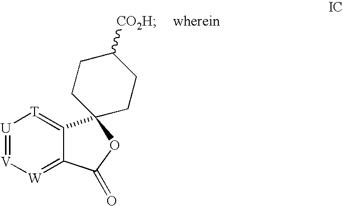
T, U, V and W are each independently selected from the group consisting of
(1) nitrogen, and
(2) methine,
wherein the methine group is unsubstituted or optionally substituted with a substituent selected from the group consisting of
   (a) halogen,
   (b) lower alkyl,
   (c) hydroxy, and
   (d) lower alkoxy, and
      wherein at least two of T, U, V, and W are methine;
comprising the steps of
(a) combining a strong base with a compound of formula A 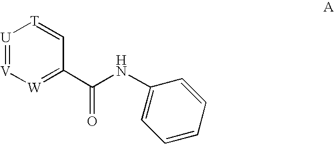
wherein T, U, V and W are as defined above, in an aprotic solvent to form a solution;
(b) reacting a compound of formula B 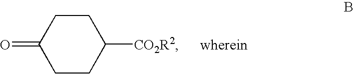
R² is selected from the group consisting of:
   (a) lower alkyl, and
   (b) —CH₂-phenyl, wherein the phenyl group is
   unsubstituted or substituted with a substituent selected from the group consisting of
      (1) lower alkyl,
      (2) lower alkoxy, and
      (3) —NO₂,
with the solution of step (a) to form an ester of formula C in solution 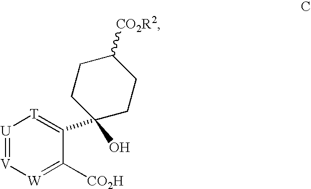
wherein T, U, V and W are as defined above;
(c) adding water to the solution of the ester of formula C in step (b) to form an acid of formula D 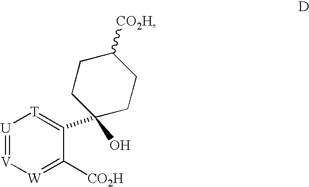
wherein T, U, V and W are as defined above;
(d) forming a spirolactone acid of formula IC 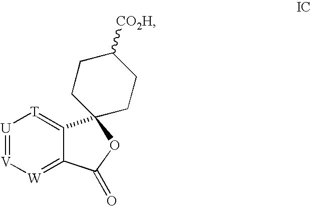
wherein T, U, V, and W are as defined above, by treating the acid of formula D with an aqueous acid;
(e) forming an spirolactone acid halide of formula E 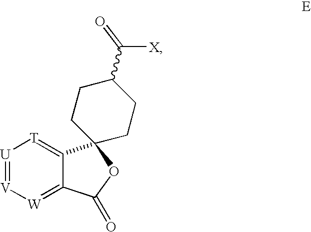
wherein X is chlorine or bromine, and T, U, V, and W are as defined above, by treating the compound of formula IC with a halogenating agent in a solvent;
(f) forming a spirolactone ester of formula F 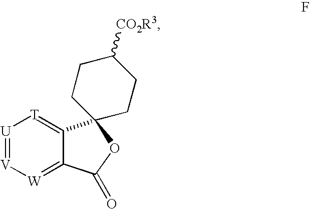
wherein R³ is selected from the group consisting of tert-butyl, methyl cyclohexyl, methyl cyclopentyl, and neopentyl, and T, U, V and W are as defined above, by treating the spirolactone acid halide of formula E with a base and an alcohol in a solvent;
(g) forming a spirolactone acid of formula IC 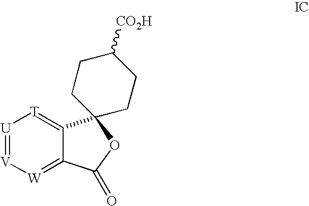
wherein T, U, V and W are defined as above, by hydrolyzing the spirolactone ester of formula F with an aqueous acid; and
(h) isolating the resulting product.

In one embodiment of the present invention, the process comprises increasing the amount of trans isomer IA 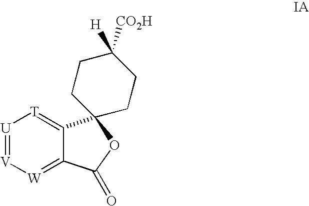
in the compound of structural formula IC 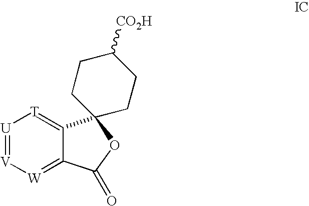
relative to the amount of cis isomer IB 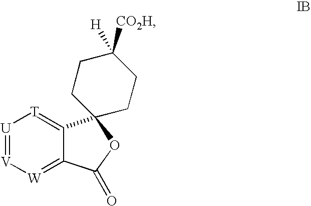
in the compound of structural formula IC,
wherein T, U, V and W are each independently selected from the group consisting of:
(1) nitrogen, and
(2) methine,
wherein the methine group is unsubstituted or optionally substituted with a substituent selected from the group consisting of:
   (a) halogen,
   (b) lower alkyl,
   (c) hydroxy, and
   (d) lower alkoxy, and
wherein at least two of T, U, V, and W are methine.

In another embodiment of the present invention, T, V and W are methine, wherein the methine group is unsubstituted or optionally substituted with a substituent selected from the group consisting of
(a) halogen,
(b) lower alkyl,
(c) hydroxy, and
(d) lower alkoxy; and
   U is nitrogen.

In a class of this embodiment, T, V and W are unsubstituted methine; and U is nitrogen.

In another embodiment of the present invention, T, U, V and W are methine, wherein the methine group is unsubstituted or optionally substituted with a substituent selected from the group consisting of
(a) halogen,
(b) lower alkyl,
(c) hydroxy, and
(d) lower alkoxy.

In one class of this embodiment, the methine group is unsubstituted or optionally substituted with halogen.

In another embodiment of the present invention, steps (a) and (b) are run at a temperature of between about −50° C. and −80° C. In a class of this embodiment, step (a) is aged at a temperature less than about −55° C. In a subclass of this class, step (a) is aged for a period between about 5 minutes to 18 hours.

In another embodiment of this invention, the aprotic solvent of step (a) is selected from the group consisting of tetrahydrofuran, toluene, heptane, dimethoxyethane, benzene, and hexane, diethyl ether, xylene, or a mixture thereof. In a class of this embodiment, the aprotic solvent of step (a) is tetrahydrofuran.

In another embodiment of this invention, the strong base of step (a) is selected from the group consisting of n-BuLi, sec-BuLi, t-BuLi, LiHMDS, NaHMDS, KHMDS and LiTMP. In a class of this embodiment, the strong base of step (a) is n-BuLi.

In another embodiment of this invention, step (a) further comprises adding a salt selected from the group consisting of LiBr, LiCl, LiI, LiBF₄, LiClO₄, and CeCl₃. In a class of this embodiment, the salt of step (a) is LiBr.

In another embodiment of this invention, R² is selected from the group consisting of: —CH₃, —CH₂CH₃, —(CH₂)₂CH₃, —CH(CH₃)₂, —(CH₂)₃CH₃, and —CH(CH₃)₃. In a class of this embodiment, R² is —CH₂CH₃.

In another embodiment of the present invention, water is added to the solution of the ester of formula C in step (c) at a temperature of about 60° C. to about −50° C. In a class of this embodiment, water is added at a temperature of about −550° C.

In another embodiment of the present invention, step (c) is run at a temperature between about 0° C. to 50° C. after the addition of water. In a class of this embodiment, step (c) is run at a temperature of about 40° C. after the addition of water. In a subclass of this class, step (c) is run for a period between about 1 hour to 4 hours.

In another embodiment of the present invention, the aqueous acid of step (d) is selected from the group consisting of hydrochloric acid, sulfuric acid, methane sulfonic acid, trifluoromethane sulfonic acid, or a mixture thereof. In a class of this embodiment, the aqueous acid of step (d) is sulfuric acid. In a subclass of this class, the acid is added at a temperature of about less than 30° C. In another subclass of this class, the acid is added at a temperature of about less than 30° C., and aged at a temperature between about 50° C. to about 70° C. for a period of about 1 hour to about 4 hours. In another embodiment of the present invention, the spirolactone acid halide of formula E in step (e) is a spirolactone acid chloride.

In another embodiment of the present invention, the solvent in step (e) is selected from the group consisting of chloroform, ethyl acetate, tetrahydrofuran, dimethoxyethane, diglyme, 2-methyl tetrahydrofuran, 1,4-dioxane and diethoxymethane. In a class of this embodiment, the solvent in step (e) is tetrahydrofuran.

In another embodiment of the present invention, the halogenating agent in step (e) is selected from the group consisting of phosphorus oxychloride, oxalyl chloride, phosphorus trichloride, phosphorus tribromide, thionyl chloride, thionyl bromide and oxalyl bromide. In a class of this embodiment, the halogenating agent in step (e) is phosphorus oxychloride. In a subclass of this class, the amount of phosphorus oxychloride is between about 0.7 equivalents to about 2.0 equivalents relative to spirolactone acid IC. In another subclass of this class, the amount of phosphorus oxychloride is about 1.15 equivalents relative to spirolactone acid IC. In another subclass of this class, the amount of phosphorus oxychloride is about 1.05 equivalents relative to spirolactone acid IC.

In another embodiment of the present invention, the reaction of step (e) further comprises a catalyst In a class of this embodiment, the catalyst is dimethyl formamide. In a subclass of this class, the amount of dimethyl formamide is between about 0.2 equivalents to about 5 equivalents relative to spirolactone acid of formula IC. In another subclass of this class, the amount of dimethyl formamide is about 1 equivalent relative to spirolactone acid of formula IC.

In another embodiment of the present invention, the reaction of step (e) is run at a temperature between about 20° C. to about 80° C. In a class of this embodiment, the reaction of step (e) is run at a temperature of about 40° C. In a subclass of this class, the reaction of step (e) is run at a temperature of about 40° C. for about 2 hours.

In another embodiment of the present invention, the base of step (f) is selected from the group consisting of N,N,N′N′-tetramethylethylenediamine, triethyl amine, N,N-diisopropylethyl amine, N,N-dimethylethyl amine, pyridine, collidine, 1,8-diazabicyclo[5.4.0]undec-7-ene, N-methylmorpholine, and N,N,N′,N′-tetramethyl-1,6-hexanediamine. In a class of this embodiment, the base of step (f) is N,N,N′,N′-tetramethylethylene-diamine. In a subclass of this class, the amount of N,N,N′,N′-tetramethylethylene-diamine is between about 1 equivalent to about 10 equivalents relative to spirolactone ester of formula F. In another subclass of this class, the amount of N,N,N′,N′-tetramethylethylene diamine is about 3.5 equivalents relative to spirolactone ester of formula F.

In another embodiment of the present invention, the alcohol of step (f) is selected from the group consisting of tert-butyl alcohol, methyl cyclohexanol, methyl cyclopentanol, and neopentyl alcohol. In a class of this embodiment, the alcohol of step (f) is tert-butyl alcohol. In a subclass of this class, the amount of tert-butyl alcohol is between about 1 equivalent to about 10 equivalents relative to spirolactone ester of formula F. In another subclass of this class, the amount of tert-butyl alcohol is about 1.5 equivalents relative to spirolactone ester of formula F.

In one embodiment of the present invention, the solvent in step (f) is selected from the group consisting of tetrahydrofuran, dimethoxyethane, diglyme, 2-methyl tetrahydrofuran, 1,4-dioxane and diethoxymethane. In a class of this embodiment, the solvent in step (f) is tetrahydrofuran.

In another embodiment, the reaction of step (f) further comprises a salt. In a class of this embodiment, the salt is selected from the group consisting of lithium bromide, lithium chloride, lithium iodide, lithium perchlorate and lithium tetrafluoroborate. In a subclass of this class, the salt is lithium chloride. In a subclass of this subclass, the amount of lithium chloride is between about 0.5 equivalents to about 5 equivalents relative to spirolactone ester of formula F. In another subclass of this subclass, the amount of lithium chloride is about 1 equivalent relative to spirolactone ester of formula F.

In another embodiment of the present invention, the reaction of step (f) is run at a temperature between about 20° C. to about 80° C. In a class of this embodiment, the reaction of step (f) is run at a temperature of about 40° C. In a subclass of this class, the reaction of step (f) is run at a temperature of about 40° C. for about 2 hours to about 24 hours. In another subclass of this class, the reaction of step (f) is run at a temperature of about 40° C. for about 19 hours.

In another embodiment of the present invention, the aqueous acid of step (g) is selected from the group consisting of sulfuric acid, hydrochloric acid, hydrobromic acid, phosphoric acid and formic acid. In a class of this embodiment, the aqueous acid of step (g) is sulfuric acid.

In another embodiment of the present invention, the hydrolysis of step (g) is run at a temperature between about 20° C. and about 100° C. In a class of this embodiment, the hydrolysis of step (g) is run at a temperature of about 50° C. In a subclass of this class, the hydrolysis of step (g) is run at a temperature of about 50° C. for about 2 hours.

In another embodiment of the present invention, the product of step (h) is isolated by adjusting the pH of the solution of step (g) to between about 0 and 4 with a base and extracting the reaction mixture to afford the compound IC. In a subclass of this class, the base is sodium hydroxide. In another subclass, the pH of step (g) is adjusted to between about about 2 to about 3. In a subclass of this subclass, the pH is adjusted to about 2.4.

By this invention, there is further provided a process for the preparation and separation of a spirolactone of formula IA, or a salt thereof, and a spirolactone of formula IB, or a salt thereof, 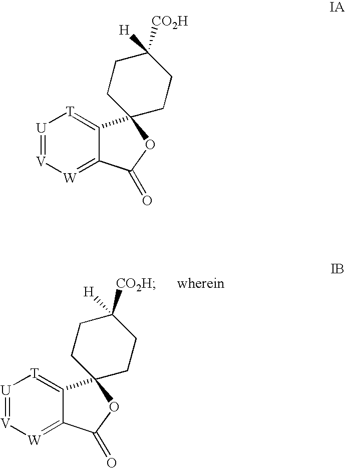
T, U, V and W are each independently selected from the group consisting of
(1) nitrogen, and
(2) methine,
wherein the methine group is unsubstituted or optionally substituted with a substituent selected from the group consisting of
   (a) halogen,
   (b) lower alkyl,
   (c) hydroxy, and
   (d) lower alkoxy, and
      wherein at least two of T, U, V, and W are methine;
      comprising the steps of
(i) adding a solvent to the compound of formula IC, 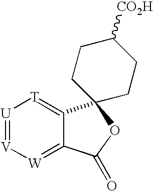
wherein T, U, V and W are as defined above, to form a mixture;
(j) adding an acid to the mixture of step (i) to form a mixture; and
(k) aging the mixture of step (j) for a time and under conditions effective to afford the compound IA 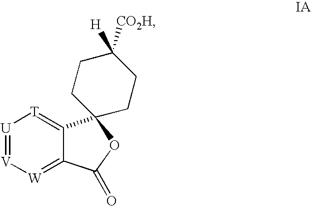
wherein T, U, V and W are as defined above, or a salt thereof.

In one embodiment of the present invention, T, V and W are methine, wherein the methine group is unsubstituted or optionally substituted with a substituent selected from the group consisting of
(a) halogen,
(b) lower alkyl,
(c) hydroxy, and
(d) lower alkoxy; and
   U is nitrogen.

In a class of this embodiment, T, V and W are unsubstituted methine; and U is nitrogen.

In another embodiment of the present invention, T, U, V and W are methine, wherein the methine group is unsubstituted or optionally substituted with a substituent selected from the group consisting of
(a) halogen,
(b) lower alkyl,
(c) hydroxy, and
(d) lower alkoxy.

In one class of this embodiment, the methine group is unsubstituted or optionally substituted with halogen.

In another embodiment of this invention, the solvent of step (i) is selected from the group consisting of dimethoxyethane, acetonitrile, tetrahydrofuran, or a mixture thereof. In a class of this embodiment, the solvent of step (i) is tetrahydrofuran. In another class of this embodiment, the solvent of step (i) is acetonitrile.

In another embodiment of this invention, the acid of step (j) is selected from the group consisting of hydrochloric acid, hydrobromic acid, tartaric acid, methane sulfonic acid, toluene sulfonic acid, succinic acid, and sulfuric acid. In a class of this embodiment, the acid of step (O) is hydrochloric acid.

In another embodiment of this invention, the step (k) is aged at a temperature of about 10° C. to 60° C. In a class of this embodiment, step (k) is aged for a period between about 1 hour to about 48 hours. In a subclass of this class, step (k) is aged at a temperature of about 25° C. for about 3 hours.

In another embodiment of this invention, the process further comprises step (1) of isolating the compound of formula IA, or a salt thereof. In a class of this embodiment, the compound of formula IA is isolated by filtering and concentrating the filtrate to give a slurry. In a subclass of this class, the slurry is diluted with a solvent and aged for a time and under conditions to give the compound of formula IA. In another subclass of this class, the slurry is diluted with hexane and aged for about 20 hours at about 0° C. In a subclass of this subclass, the compound of formula IA is isolated by filtering the slurry to give the product. In another subclass of this class, the slurry is concentrated, diluted with acetonitrile and aged for a time and under conditions to give the compound of formula IA.

In another embodiment of this invention, there is provided a compound of structural formula, or a salt thereof, 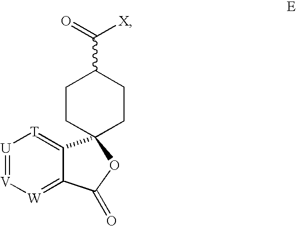
wherein X is selected from the group consisting of chlorine and bromine, and
T, U, V and W are each independently selected from the group consisting of:
(1) nitrogen, and
(2) methine,
wherein the methine group is unsubstituted or optionally substituted with a substituent selected from the group consisting of:
   (a) halogen,
   (b) lower alkyl,
   (c) hydroxy, and
   (d) lower alkoxy, and
      wherein at least two of T, U, V, and W are methine.

In one class of this embodiment, T, V and W are methine, wherein the methine group is unsubstituted or optionally substituted with a substituent selected from the group consisting of
(a) halogen,
(b) lower alkyl,
(c) hydroxy, and
(d) lower alkoxy; and
   U is nitrogen.

In a subclass of this class, T, V and W are unsubstituted methine; and U is nitrogen.

In another class of this embodiment, T, U, V and W are methine, wherein the methine group is unsubstituted or optionally substituted with a substituent selected from the group consisting of
(a) halogen,
(b) lower alkyl,
(c) hydroxy, and
(d) lower alkoxy.

In a subclass of this class, the methine group is unsubstituted or optionally substituted with halogen.

In another embodiment of this invention, there is provided a compound of structural formula 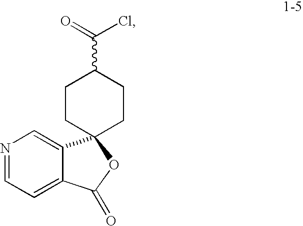
or a salt thereof.

In another embodiment of this invention, there is provided a composition comprising about 83% to 52% of compound IA 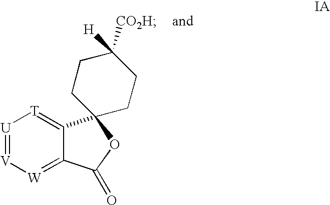
about 17% to 48% of compound IB 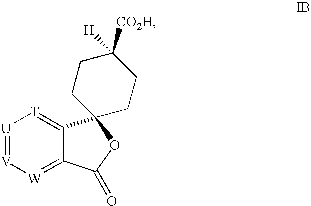
T, U, V and W are each independently selected from the group consisting of:
(1) nitrogen, and
(2) methine,
wherein the methine group is unsubstituted or optionally substituted with a substituent selected from the group consisting of:
   (a) halogen,
   (b) lower alkyl,
   (c) hydroxy, and
   (d) lower alkoxy, and
      wherein at least two of T, U, V, and W are methine.

In one class of this embodiment, T, V and W are methine, wherein the methine group is unsubstituted or optionally substituted with a substituent selected from the group consisting of
(a) halogen,
(b) lower alkyl,
(c) hydroxy, and
(d) lower alkoxy; and
   U is nitrogen.

In a subclass of this class, T, V and W are unsubstituted methine; and U is nitrogen.

In another class of this embodiment, T, U, V and W are methine, wherein the methine group is unsubstituted or optionally substituted with a substituent selected from the group consisting of
(a) halogen,
(b) lower alkyl,
(c) hydroxy, and
(d) lower alkoxy.

In a subclass of this class, the methine group is unsubstituted or optionally substituted with halogen.

In another embodiment of this invention, there is provided a composition comprising about 79% of compound 1-8 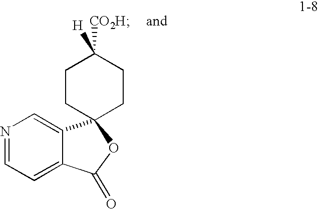
about 21% of compound 1-9

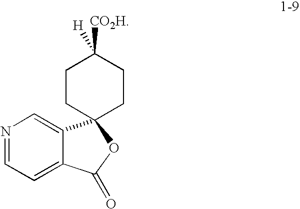

In yet another embodiment of this invention, there is provided a composition comprising about 83% of compound 1-8 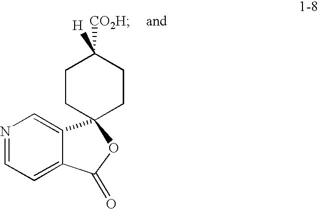
about 17% of compound 1-9

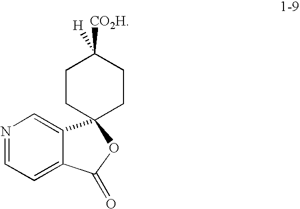

As used herein “T, U, V and W” refer to a nitrogen or a methine, wherein the methine group is unsubstituted or optionally substituted with a substituent selected from the group consisting of halogen, lower alkyl, hydroxy, and lower alkoxy, and wherein at least two of T, U, V, and W are methine.

“Methine group is unsubstituted or optionally substituted with a substituent selected from the group consisting of halogen, lower alkyl, hydroxy and lower alkoxy” refers to unsubstituted methine or methine having a substituent which can be selected from the group consisting of halogen, lower alkyl, hydroxy and lower alkoxy. The aforesaid substituent includes preferably halogen, and the like.

“Halogen” or “halide” refers to fluorine atom, chlorine atom, bromine atom and iodine atom. Halogen atom as the aforesaid substituent includes preferably fluorine atom, chlorine atom, and the like.

“Lower alkyl” refers to a straight- or branched-chain alkyl group of C₁ to C₆, for example, methyl, ethyl, propyl, isopropyl, butyl, isobutyl, sec-butyl, tert-butyl, pentyl, isopentyl, hexyl, isohexyl, and the like. Lower alkyl as the aforesaid substituent includes preferably methyl, ethyl, and the like.

“Lower alkoxy” refers to a straight- or branched-chain alkoxy group of C₁ to C₆, for example, methoxy, ethoxy, propoxy, isopropoxy, butoxy, sec-butoxy, isobutoxy, tert-butoxy, pentyloxy, isopentyloxy, hexyloxy, isohexyloxy, and the like. Lower alkoxy as the aforesaid substituent includes preferably methoxy, ethoxy, and the like.

“Cycloalkyl” refers to a monocyclic saturated carbocyclic ring of C₃ to C₆, wherein one carbocyclic ring carbon is the point of attachment. Examples of cycloalkyl include, but are not limited to, cyclopropyl, cyclobutyl, cyclopentyl, cyclohexyl, and the like.

“Cycloheteroalkyl” refers to a monocyclic saturated ring containing at least one heteroatom selected from N, S and O of C₃ to C₆, in which the point of attachment may be carbon or nitrogen. Examples of “cycloheteroalkyl” include, but are not limited to, pyrrolidinyl, piperidinyl, piperazinyl, imidazolidinyl, tetrahydrofuranyl, morpholinyl, and the like.

“Aryl” refers to a mono- or bicyclic aromatic rings containing only carbon atoms. The term also includes aryl group fused to a monocyclic cycloalkyl or monocyclic cycloheteroalkyl group in which the point of attachment is on the aromatic portion. Examples of aryl include phenyl, naphthyl, indanyl, indenyl, tetrahydronaphthyl, 2,3-dihydrobenzofuranyl, dihydrobenzopyranyl, 1,4-benzodioxanyl, and the like. The aryl ring may be unsubstituted or substituted on one or more carbon atoms.

“Heteroaryl” refers to a mono- or bicyclic aromatic ring, wherein each ring has 5 or 6 carbons, containing at least one heteroatom selected from N, O and S. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl, and the like. The heteroaryl ring may be unsubstituted or substituted on one or more carbon atoms.

As used herein, the term “anion” refers to a mono-anion or a di-anion.

The compounds in the processes of the present invention include stereoisomers, diastereomers and geometerical isomers, or tautomers depending on the mode of substitution. The compounds may contain one or more chiral centers and occur as racemates, racemic mixtures and as individual diastereomers, diastereomeric mixtures, enantiomeric mixtures or single enantiomers, or tautomers. The present invention is meant to comprehend all such isomeric forms of the compounds in the compositions of the present invention, and their mixtures. Therefore, where a compound is chiral, the separate enantiomers, and diastereomers, substantially free of the other, are included within the scope of the invention; further included are all mixtures of enantiomers, and all of the mixtures of diastereomers. Also included within the scope of the invention are salts, polymorphs, hydrates and solvates of the compounds and intermediates of the instant invention.

Compounds of the structural formula I and structural formula II include stereoisomers, such as the trans-form of compounds of the general formulas IA and IIA: 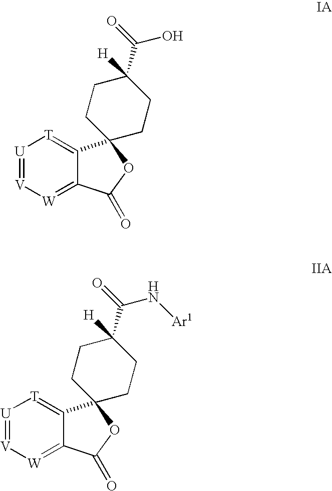
and the cis-form compounds of the general formula IB and IIB: 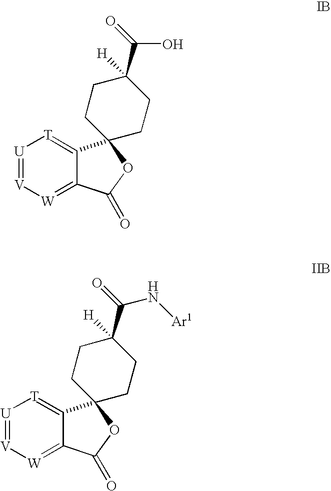
The trans form is preferred.

The salts of compounds of formula I, IA, IB, and IC refer to the pharmaceutically acceptable and common salts, for example, base addition salt to carboxyl group when the compound has a carboxyl group, or acid addition salt to amino or basic cycloheteroalkyl when the compound has an amino or basic cycloheteroalkyl group, and the like.

The base addition salts include salts with alkali metals (including, but not limited to, sodium, potassium); alkaline earth metals (including, but not limited to, calcium, magnesium); ammonium or organic amines (including, but not limited to, trimethylamine, triethylamine, dicyclohexylamine, ethanolamine, diethanolamine, triethanolamine, procaine, N,N′-dibenzylethylenediamine), and the like.

The acid addition salts include salts with inorganic acids (including, but not limited to, hydrochloric acid, sulfuric acid, nitric acid, phosphoric acid, perchloric acid), organic acids (including, but not limited to, maleic acid, fumaric acid, tartaric acid, citric acid, ascorbic acid, trriluoroacetic acid, acetic acid), sulfonic acids (including, but not limited to, methanesulfonic acid, isethionic acid, benzenesulfonic acid, p-toluenesulfonic acid, p-toluenesulfonic acid monohydrate, p-toluene sulfonic acid hydrate, camphor sulfonic acid), and the like.

In the schemes and examples below, various reagent symbols and abbreviations have the following meanings:
n-BuLi or BuLi: n-butyl lithium
sec-BuLi: sec-butyl lithium
t-BuLi: tert-butyl lithium
t-BuOH: tert-butyl alcohol
DBU: 1,8-diazabicyclo[5.4.0]undec-7-ene
DMF: dimethyl formamide
DMSO: dimethyl sulfoxide
-Et: —CH₂CH₃
g: grams
h: hours
HCl: hydrochloric acid
H₂SO₄: sulfuric acid
KHMDS: potassium hexamethyl disilazide
LiBr: lithium bromide
LiCl: lithium chloride
LiHMDS: lithium hexamethyl disilazide
LiTMP: lithium tetramethyl piperadide
NaHMDS: sodium hexamethyl disilazide
-Me: methyl
mL: milliliter
mmol: millimole
mol: moles/liter
POCl₃: phosphorus oxychloride
THF: tetrahydrofuran
TMEDA tetramethylethylenediamine or N,N,N′,N′-tetramethylethylenediamine

The compounds of the present invention can be prepared by employing the general process in Scheme 1. The novel process of the present invention can be exemplified in Scheme 2, which illustrates the preparation of the spirolactones of structural formula I, IA, IB and IC, and salts thereof. The salts of IA and IB may be separated and individually reacted with an amine, H₂NAr¹. For example, the neutralization, activation and subsequent reaction of the salt of IA with H₂NAr¹ yields compounds of formula II.

In Scheme 2, the 4-ethyl ester substituted cyclohexanone is converted to the carboxylic acid before ring lactonization to form the spirolactone IC, via intermediate C. Isonicotinamide 1-1 is deprotonated with a base, such as n-butyllithium, in the presence of a salt, such as LiBr, in a solvent such as THF, and at a temperature between about −55° C. to −65° C., to form a metallated anilide. The metallated anilide is added to a solution of ethyl 4-oxocyclohexanecarboxylate 1-2 in a solvent such as THF, at a temperature below about −55° C., followed by the addition of water to form the diacid 1-3. The diacid 1-3 is then treated with an aqueous acid, such as sulfuric acid, at a temperature below about 30° C., to form the lactone ring of spirolactone acid 14, as a mixture of about 1:1 cis to trans spirolactone acids. Spirolactone acid 14 is then activated by forming an acid halide 1-5, by treatment with a halogenating agent in a solvent such as THF in the presence of DMF. The acid halide is preferentially an acid chloride formed by treatment of the acid with phosphorus oxychloride. The acid chloride 1-5 is treated with a base such as N,N,N′,N-tetramethylethylenediamine, in the presence of an alcohol, such as tert-butanol, and a salt, such as LiCl, in a solvent such as THF, to form an ester 1-6 via a ketene intermediate. The ester 1-6 is subsequently hydrolyzed with an aqueous acid, such as aqueous sulfuric acid, at a temperature of about 50° C., to form acid 1-7 (IC) as a 80:20 trans/cis mixture. The acid 1-7 may be further purified and separated into acids 1-8 (IA, trans) and 1-9 (IB, cis) by forming a salt of 1-9 with an acid, such as hydrochloric acid, and separating the compounds by recrystallizing from a solvent such as acetonitrile, tetrahydrofuran, heptane or a mixture thereof. This process provides IA substantially free from IIB and provides IB substantially free from IA.

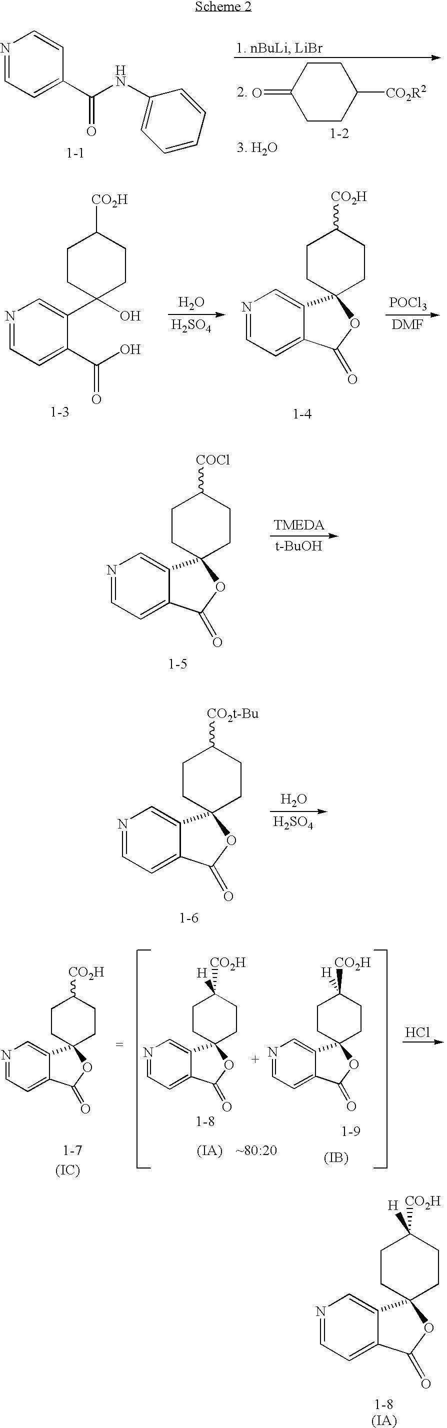

The following examples are provided to illustrate the invention and are not to be construed as limiting the scope of the invention in any manner.

### EXAMPLE 1

### Preparation of Trans-1′-oxospiro[cyclohexane-1,3′(1′H)-furo[3,4-C]pyridine]-4-carboxylic acid, 1-5, (Method A)

Step A: Preparation of Compound 1-3

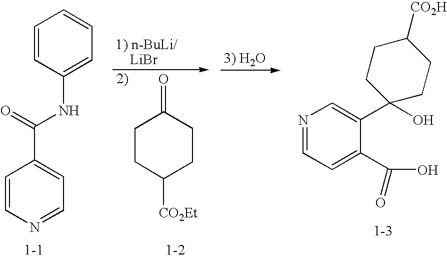

The isonicotinamide 1-1 (100 g, 0.50 mol, Kingchem), THF (0.5 L) and a 1 M LiBr solution (prepared by dissolving 1.50 mol of LiBr in 1.5 L of THF) were mixed in a flask. The resulting solution was degassed with nitrogen and cooled to −65° C. n-BuLi (1.56 M in hexane; 666 mL, 1.04 mol) was then added while maintaining the batch temperature below −55° C. The resulting solution was then aged at a temperature less than −55° C. for a period between 1 to 7 hours to give a metalated anilide mixture.

A solution of ethyl 4-oxocyclohexanecarboxylate 1-2 (100 mL, 0.63 mol, EMS Dottikon AG) in THF (1 L) was cooled in a separate flask to a temperature below −60° C. To the solution was added the above metalated anilide mixture, while maintaining the batch temperature below −55° C. The resulting solution was aged at a temperature below −55° C. for 1 hour and then carefully quenched into H₂O (1 L). The resulting mixture was warmed to 40° C. and aged at 40° C. for a period between 1 to 4 hours. After cooling to room temperature, the organic layer was removed and the aqueous layer (1.3 L; pH ˜11) was washed with THF (1 L) to give an aqueous solution of the diacid 1-3.
Step B: Preparation of Compound 1-4

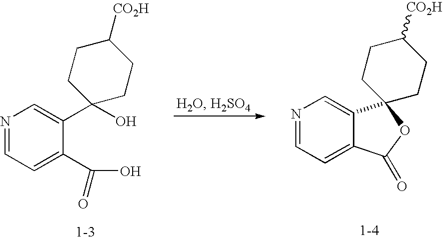

To the aqueous solution of the diacid 1-3 from Step A was added H₂O (500 mL, 5 mL/g of anilide) and 47% aqueous H₂SO₄ to adjust to pH 2-3, maintaining the temperature below 30° C. The resulting white suspension was aged at a temperature of 30° C.-70° C. for a period of 1 to 4 hours. After cooling the batch, THF (2500 mL) and 20% aqueous NaCl (600 ml) were added to extract the product acid 1-4. After the separation of the two layers, the water layer was re-extracted with THF (1000 mL). The combined THF extracts (3500 mL) were concentrated to 1250 mL. The mixture turned to a suspension of spirolactone acid 1-4 during the distillation.

Selected Signals: ¹H NMR (300.13 MHz, DMSO-d₆): Λ 12.31 (br, 1H), 9.10 (d, 1H), 8.85 (m, 1H), 7.82 (m, 1H). 2.70 (m, 0.45H), 2.43 (m, 0.55H), 1.65-2.25 (m, 8H).
Step C: Preparation of Compound 1-7

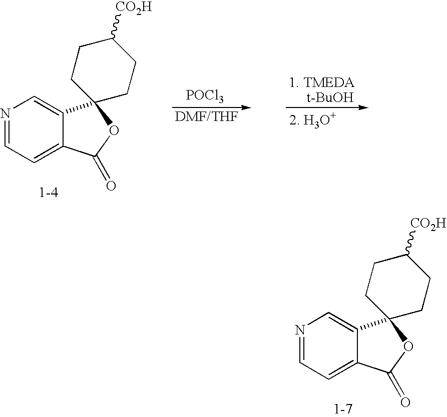

Spirolactone acid 1-4 (800 g of a 55 A % cis:45 A % trans mixture) was added to a 50 L vessel containing THF (17.6 L). The slurry was treated with DMF (260 mL, 3.2 mol) and then at 22° C., with POCl₃ (350 mL) over 10 min to form the acid chloride 1-5. The solution was warmed to 40° C. over 45 min, aged for 2 h and then cooled to 24° C. In a separate 12 L flask was sequentially added: THF (3.3 L), TMEDA (1.7 L), t-butanol (465 mL) and LiCl (143 g). After aging at 25° C. for 1 h, this resulting solution was added to the solution of acid chloride 1-5 at 24-30° C. over 25 min and aged for 19 h at 35-39° C. The reaction mixture was cooled to 0° C. and quenched by adding 4.2 L 33% H₂SO₄ slowly over 20 min during which time the internal temperature rose to 22° C. The resulting solution was heated to 50° C. for 3 h. The solution was then cooled to 22° C. and pH adjusted to 2.4 with 6 N NaOH (7.0 kg). The organic layer was separated and washed with 2×8 L of aqueous HCl/NaCl (pH 2.5). THF (3.3 L) was added to the organic layer to raise the solution volume to about 26 L and it was charged to a 50 L flask. The organic layer was azeotropically dried via a constant volume distillation at atmospheric pressure until the KF was 0.3%. (Utilized about 51 kg THF) to provide a solution of spirolactone acid 1-7.
Step D: Separation of Compound 1-7 into Compounds 1-8 and 1-9

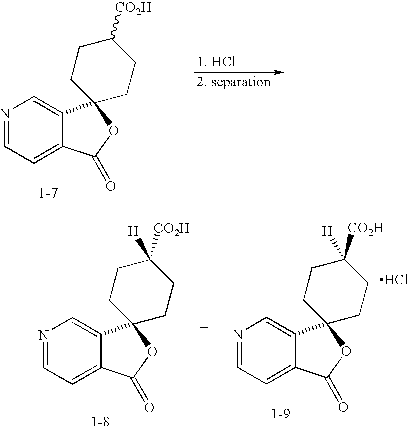

The solution of spirolactone 1-7 was cooled to 22° C. and concentrated HCl (60 mL) was slowly added to the solution. The resulting slurry was aged at 25° C. for 3 h, and the precipitate was removed via filtration and washed with THF (1×1 L). The filtrate containing spirolactone acid 1-8 was concentrated to 6.5 L in vacuo (internal temp=38-42° C.), and the resulting slurry was cooled to 22° C. over 1 h and aged for 1 h. Heptane (6 L) was added over 2 h and the slurry was cooled 0° C. and aged for 20 h, followed by vacuum filtration, rinsing the product cake with THF-heptane (2/3; 2×600 mL) and drying in vacuo at 45° C. to provide the spirolactone acid 1-8.

¹H NMR (400.13 MHz; DMSO-d₆): A 12.34 (br, 1H), 9.04 (d, J=1.0 Hz, 1H), 8.85 (d, J=5.0 Hz, 1H), 7.82 (dd, J=5.0 Hz, 1.0 Hz, 1H), 2.70 (br m, 1H), 2.08-1.89 (overlapping m, 6H), 1.82-1.76 (overlapping m, 2H).

¹³C NMR (100.62 MHz; DMSO-d₆): 175.9, 167.9, 150.6, 147.5, 144.9, 133.1, 119.1, 87.2, 38.1, 33.1, 23.9.

Alternatively, spirolactone 1-8 may be crystallized from acetonitrile according to the following procedure. The filtrate containing spirolactone acid 1-8 in step D (250 ml; 15 g/L trans Acid) was concentrated to 44 ml via distillation and cooled to 40° C. Acetonitrile (7.5 mL) was added with 50 mg seed. The slurry was aged at 40° C. for 2.5 h, cooled to 22° C. and aged for 2 h. The remaining THF was removed by a constant volume distillation feeding in acetonitrile until the THF level was <2 A %. The batch was cooled to 0° C. and aged for 2 hours prior to filtration, then washed with chilled acetonitrile (1×10 mL), and dried in vacuo to give spirolactone acid 1-8.

While the invention has been described and illustrated with reference to certain particular embodiments thereof, those skilled in the art will appreciate that various changes, modifications and substitutions can be made therein without departing from the spirit and scope of the invention. It is intended, therefore, that the invention be defined by the scope of the claims which follow and that such claims be interpreted as broadly as is reasonable.

What is claimed is:

1. A process for preparing a compound of the formula IC, or a salt thereof, 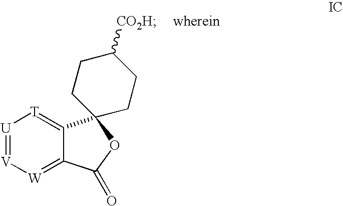
T, U, V and W are each independently selected from the group consisting of:
(1) nitrogen, and
(2) methine,
wherein the methine group is unsubstituted or optionally substituted with a substituent selected from the group consisting of:
(a) halogen,
(b) lower alkyl,
(c) hydroxy, and
(d) lower alkoxy, and
wherein at least two of T, U, V, and W are methine;
comprising the steps of:
(a) forming an spirolactone acid halide of formula E 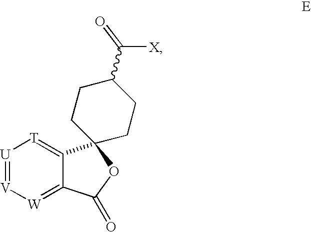
wherein X is chlorine or bromine, and T, U, V, and W are as defined above, by treating the compound of formula IC with a halogenating agent in a solvent;
(b) forming a spirolactone ester of formula F 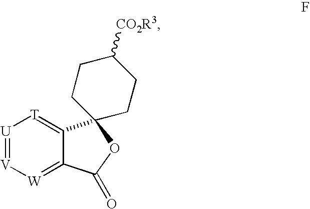
wherein R³ is selected from the group consisting of tert-butyl, methyl cyclohexyl, methyl cyclopentyl, and neopentyl, and T, U, V and W are as defined above, by treating the spirolactone acid halide of formula E with a base and an alcohol in a solvent;
(c) forming a spirolactone acid of formula IC 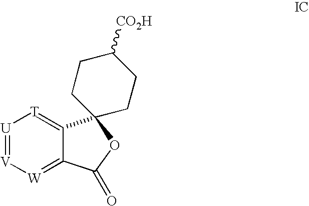
wherein T, U, V and W are defined as above, by hydrolyzing the spirolactone ester of formula F with an aqueous acid; and
(d) isolating the resulting product.

2. The process of claim 1 wherein the solvent of step (a) is selected from the group consisting of chloroform, ethyl acetate, tetrahydrofuran, dimethoxyethane, diglyme, 2-methyl tetrahydrofuran, 1,4-dioxane and diethoxymethane.

3. The process of claim 2 wherein the solvent of step (a) is tetrahydrofuran.

4. The process of claim 1 wherein the halogenating agent in step (a) is selected from the group consisting of phosphorus oxychloride, oxalyl chloride, phosphorus trichloride, phosphorus tribromide, thionyl chloride, thionyl bromide and oxalyl bromide.

5. The process of claim 4 wherein the halogenating agent in step (a) is phosphorus oxychloride.

6. The process of claim 1 wherein the spirolactone acid halide of formula E in step (a) is a spirolactone acid chloride.

7. The process of claim 1 wherein step (a) further comprises a catalyst.

8. The process of claim 7 wherein the catalyst of step (a) is dimethyl formamide.

9. The process of claim 1 wherein the base of step (b) is selected from the group consisting of N,N,N′,N′-tetramethylethylenediamine, triethyl amine, N,N-diisopropylethyl amine, N,N-dimethylethyl amine, pyridine, collidine, 1,8-diazabicyclo[5.4.0]undec-7-ene, N-methylmorpholine, and N,N,N′,N′-tetramethyl-1,6-hexanediamine.

10. The process of claim 9 wherein the base of step (b) is N,N,N′,N′-tetramethylethylenediamine.

11. The process of claim 1 wherein the alcohol of step (b) is selected from the group consisting of tert-butyl alcohol, methyl cyclohexanol, methyl cyclopentanol, and neopentyl alcohol.

12. The process of claim 11 wherein the alcohol of step (b) is tert-butyl alcohol.

13. The process of claim 1 wherein the solvent in step (b) is selected from the group consisting of tetrahydrofuran, dimethoxyethane, diglyme, 2-methyl tetrahydrofuran, 1,4-dioxane and diethoxymethane.

14. The process of claim 13 wherein the solvent in step (b) is tetrahydrofuran.

15. The process of claim 1 wherein step (b) further comprises a salt.

16. The process of claim 15 wherein the salt of step (b) is selected from the group consisting of lithium bromide, lithium chloride, lithium iodide, lithium perchlorate and lithium tetrafluoroborate.

17. The process of claim 16 wherein the salt of step (b) is lithium chloride.

18. The process of claim 1 wherein the aqueous acid of step (c) is selected from the group consisting of sulfuric acid, hydrochloric acid, hydrobromic acid, phosphoric acid and formic acid.

19. The process of claim 18 wherein the aqueous acid of step (c) is is sulfuric acid.

20. The process of claim 1 further comprising the steps of
(e) adding a solvent to the compound of formula IC, 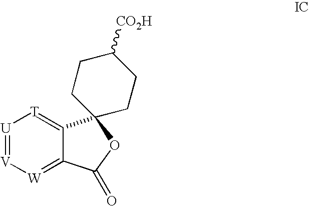
wherein T, U, V and W are as defined above, to form a mixture;
(f) adding an acid to the mixture of step (e) to form a mixture; and
(g) aging the mixture of step (f) for a time and under conditions effective to afford the compound IA 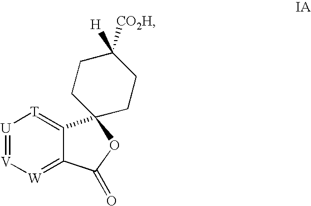
wherein T, U, V and W are as defined above, or a salt thereof.

21. The process of claim 20 wherein the solvent of step (e) is selected from the group consisting of dimethoxyethane, acetonitrile, tetrahydrofuran, or a mixture thereof.

22. The process of claim 21 wherein the solvent of step (e) is tetrahydrofuran.

23. The process of claim 20, wherein the acid of step (f) is selected from the group consisting of hydrochloric acid, hydrobromic acid, tartaric acid, methane sulfonic acid, toluene sulfonic acid, succinic acid, and sulfuric acid.

24. The process of claim 23 wherein the acid of step (f) is hydrochloric acid.

25. The process of claim 20, wherein step (g) is aged at a temperature of about 10° C. to 60° C.

26. The process of claim 25, wherein step (g) is aged at a temperature of about 25° C. for about 3 hours.

27. The process of claim 20 further comprising the step (h) of isolating the compound of formula IA, or a salt thereof.

28. The process of claim 1 wherein T, V and W are methine,
wherein the methine group is unsubstituted or optionally substituted with a substituent selected from the group consisting of
(a) halogen,
(b) lower alkyl,
(c) hydroxy, and
(d) lower alkoxy; and
U is nitrogen.

29. The process of claim 29 wherein T, V and W are unsubstituted methine; and U is nitrogen.

30. The process of claim 1 wherein T, U, V and W are methine, wherein the methine group is unsubstituted or optionally substituted with a substituent selected from the group consisting of
(a) halogen,
(b) lower alkyl,
(c) hydroxy, and
(d) lower alkoxy.

31. The process of claim 1 wherein the amount of trans isomer IA 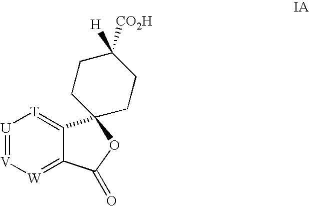
in the compound of structural formula IC 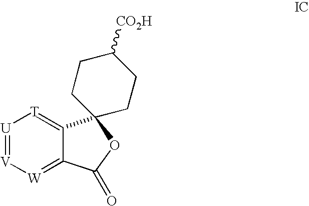
is increased relative to the amount of cis isomer IB 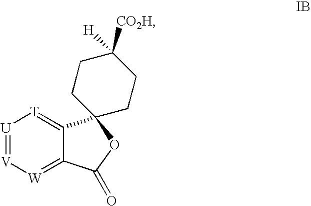
in the compound of structural formula IC,
wherein T, U, V and W are each independently selected from the group consisting of:
(1) nitrogen, and
(2) methine,
wherein the methine group is unsubstituted or optionally substituted with a substituent selected from the group consisting of:
(a) halogen,
(b) lower alkyl,
(c) hydroxy, and
(d) lower alkoxy, and
wherein at least two of T, U, V, and W are methine.

32. A composition comprising about 83% to 52% of compound IA 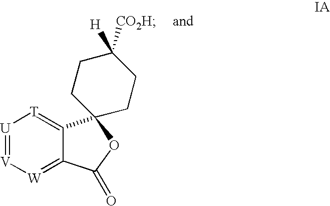
about 17% to 48% of compound IB 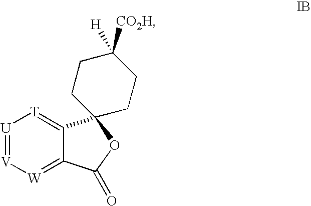
wherein T, U, V and W are each independently selected from the group consisting of:
(1) nitrogen, and
(2) methine,
wherein the methine group is unsubstituted or optionally substituted with a substituent selected from the group consisting of:
(a) halogen,
(b) lower alkyl,
(c) hydroxy, and
(d) lower alkoxy, and
wherein at least two of T, U, V, and W are methine.

33. The composition of claim 32 comprising about 79% of compound 1-8 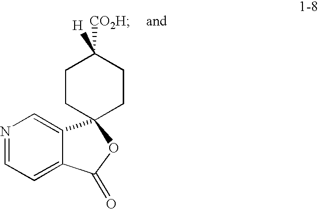
about 21% of compound 1-9

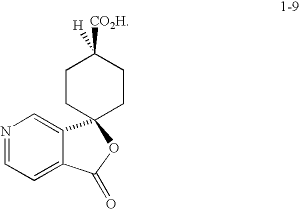

34. The composition of claim 32 comprising about 83% of compound 1-8 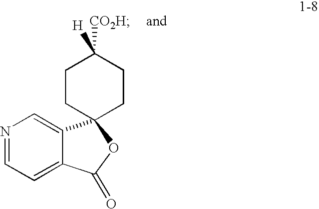
about 17% of compound 1-9 [chemical structure]
